(19) United States
(12) Patent Application Publication
(10) Pub. No.: US 2007/0238755 A1
(43) Pub. Date: Oct. 11, 2007

(54) Methods to treat and/or prevent radiation- and/or chemical-induced toxicity in non-malignant tissue

(76) Inventors: Martin Hauer-Jensen, Little Rock, AR (US); Chen Zong, Metuchen, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/642,505

(22) Filed: Dec. 20, 2006

Related U.S. Application Data
(60) Provisional application No. 60/751,820, filed on Dec. 20, 2005.

(51) Int. Cl. A61K 31/44; A61P 39/00

(52) U.S. Cl. 514/337

(57) ABSTRACT

The present invention relates to methods useful for treating and/or preventing radiation- and/or chemical-induced toxicity in non-malignant tissue using a protease activated receptor-1 (PAR-1) inhibitor. In particular, use of a protease activated receptor-1 (PAR-1) inhibitor to treat and/or prevent acute and chronic adverse effects of radiation and/or chemical exposure (e.g., to one or more of the following: intestine, lung, oral mucosa, or other organs).

### REFERENCE TO RELATED APPLICATION

This application claims the benefit of priority to U.S. Provisional Patent Application 60/751,820 filed Dec. 20, 2005, the entire disclosure of the priority application is hereby incorporated by reference.

### FIELD OF THE INVENTION

The present invention relates to methods useful for treating and/or preventing radiation- and/or chemical-induced toxicity in non-malignant tissue. In particular, use of a protease activated receptor-1 (PAR-1) inhibitor to treat and/or prevent acute and chronic adverse effects of radiation and/or chemical exposure (e.g., to one or more of the following: intestine, lung, oral mucosa, and other organs).

### BACKGROUND OF THE INVENTION

Radiation- and/or chemical-induced toxicity in non-malignant tissues may result in debilitating side effects (e.g., intestinal radiation toxicity, pneumonitis, and mucositis). Therapeutic radiation exposure, for example, utilized in bone marrow transplant and more than half of all cancer patients, plays a critical role in approximately 25% of cancer cures. In spite of advances in the ability to deliver localized radiation for the treatment of cancer, radiation toxicity in non-malignant tissue remains the most important dose-limiting factor in clinical radiation toxicity. Moreover, patients suffering from long-term side effects of radiation, such as, intestinal radiation toxicity (i.e., radiation enteropathy) have a poor long term prognosis even if they are cured of the malignancy for which they received radiation treatment. Likewise, radiation-induced pneumonitis is a familiar complication of therapeutic radiation exposure of tumors in and around the chest (e.g., breast cancer, lung cancer, and esophageal cancer). The adverse side effects associated with such therapeutic regimens interfere with the ability of patients to continue on a therapeutic regimen and oftentimes result in dose reduction or dose interruption.

Among other radiation- and/or chemical-induced toxicity in non-malignant tissues, mucositis is a common and potentially serious side effect. In fact, oral mucositis has been identified as the most debilitating side effect of anticancer therapy by patients who experienced it while undergoing myelotoxic therapy for hematopoietic stem cell transplantation. Patients suffering from severe oral mucositis may find daily activities such as eating, drinking, swallowing, and talking difficult or impossible. In oral mucositis, the degree of injury to mucosal tissue is directly related to the type dose, or dose intensity of the radiotherapy and/or chemotherapy regimens employed. When treated to ameliorate and/or prevent radiation- and/or chemical-induced toxicity in non-malignant tissues, patients receiving therapeutic radiation and/or chemical treatments may experience a higher quality of life and thereby remain on their therapeutic regimen so that the therapeutic effect may be achieved or possibly receive a more demanding and more effective therapeutic regimen.

Similarly, non-therapeutic radiation and/or chemical exposure, as may happen from accidents, acts of war, acts of civilian terrorism, space flights, or rescue and clean-up operations results in radiation- and/or chemical-induced toxicity in non-malignant tissue. In these scenarios the effects of radiation in the hematopoietic system and the gastrointestinal tract are critical. Survival after radiation exposure may be improved by minimizing the adverse effects of ionizing radiation using thrombin inhibitors. However, thrombin inhibitors (e.g., hirudin), while blocking thrombin's procoagulant, proinflammatory and fibroproliferative effects, also block important physiological responses for mitigating radiation toxicity (Wang et al., J Thromb Haemost, 2(11):2027-2035 (2004)).

When treated to ameliorate and/or prevent radiation- and/or chemical-induced toxicity in non-malignant tissues, patients may experience a higher quality of life and achieve a better clinical outcome. Thus, the need exists for methods of treating nor preventing radiation- and/or chemical-induced toxicity in non-malignant tissues as may result from therapeutic or non-therapeutic radiation and/or chemical exposure.

### SUMMARY OF THE INVENTION

The present invention provides methods useful for treating and/or preventing radiation- and/or chemical-induced toxicity in non-malignant tissue in a patient comprising administering a therapeutically effective amount of a protease activated receptor-1 (PAR-1) inhibitor. In one embodiment, the PAR-1 inhibitor is:

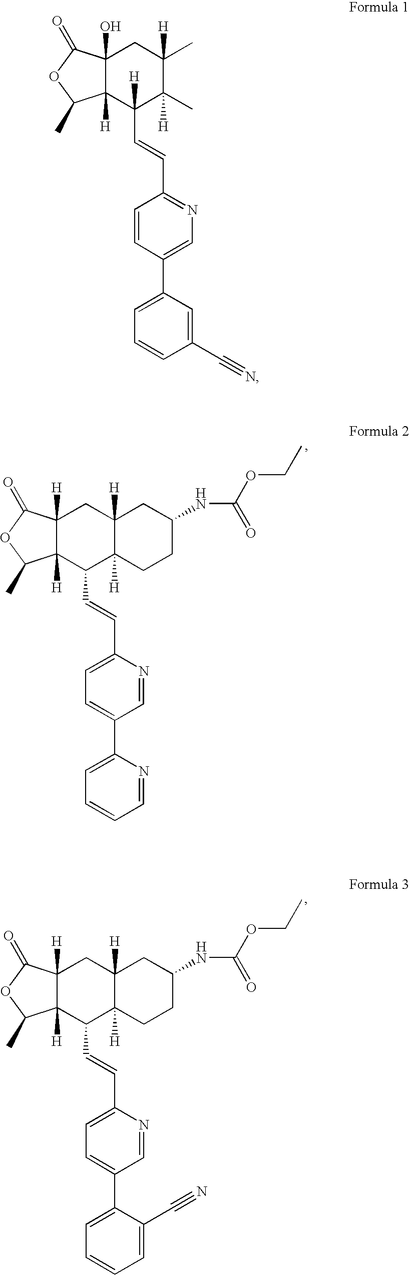

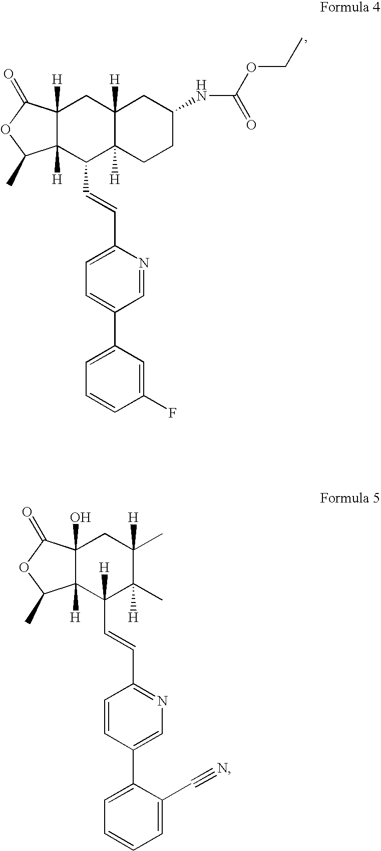

BMS-200261, RWJ-5610, RWJ-58259, a blocking antibody to PAR-1, a pepducin to PAR-1, an antisense oligonucleotide to PAR-1, a small interfering RNA or a short hairpin RNA to the mRNA encoding PAR-1, or a pharmaceutically acceptable salt thereof, or a combination of two or more of the above.

In another embodiment, the PAR-1 inhibitor is: 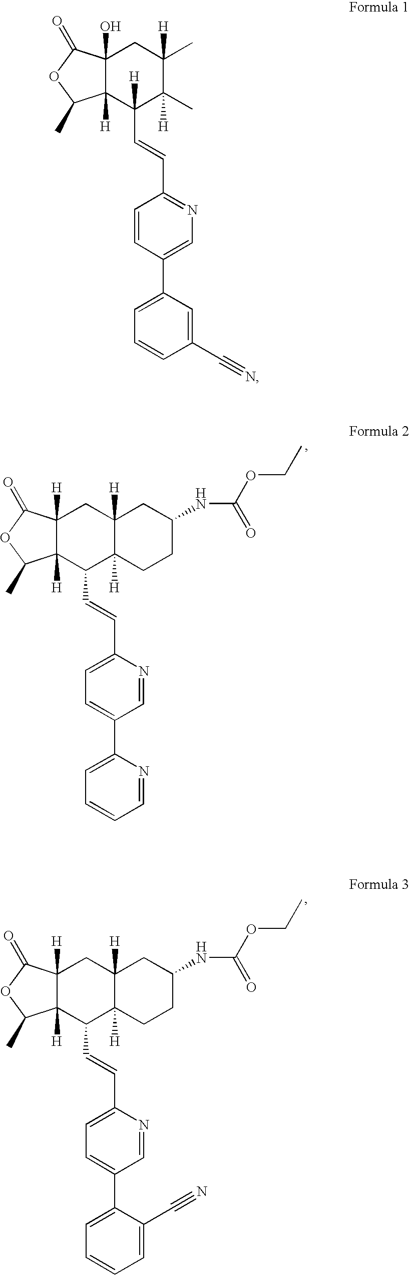 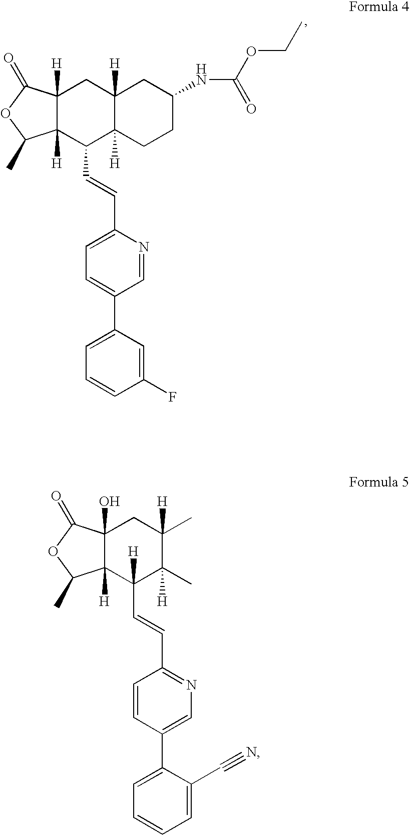
or a pharmaceutically acceptable salt thereof, or a combination of two or more of the above. In a preferred embodiment, the PAR-1 inhibitor is Formula 2, or a pharmaceutically acceptable salt thereof.

In another embodiment, the radiation- and/or chemical-induced toxicity is one or more of intestinal fibrosis, pneumonitis, and mucositis. In a preferred embodiment, the radiation- and/or chemical-induced toxicity is intestinal fibrosis. In another preferred embodiment, the radiation- and/or chemical-induced toxicity is oral mucositis. In yet another embodiment, the radiation- and/or chemical-induced toxicity is intestinal mucositis, intestinal fibrosis, intestinal radiation syndrome, or pathophysiological manifestations of intestinal radiation exposure.

In another embodiment, the PAR-1 inhibitor is administered in combination with Kepivance™ (palifermin), L-glutamine, teduglutide, sucralfate mouth rinses, iseganan, lactoferrin, mesna, trefoil factor, or a combination of two or more of the above.

In another embodiment, the PAR-1 inhibitor is administered in combination with another radiation-response modifier.

The present invention also provides methods for reducing structural radiation injury in a patient that will be exposed, is concurrently exposed, or was exposed to radiation and/or chemical toxicity, comprising administering a therapeutically effective amount of a PAR-1 inhibitor.

The present invention also provides methods for reducing inflammation in a patient that will be exposed, is concurrently exposed, or was exposed to radiation and/or chemical toxicity, comprising administering a therapeutically effective amount of a PAR-1 inhibitor.

The present invention also provides methods for adverse tissue remodeling in a patient that will be exposed, is concurrently exposed, or was exposed to radiation and/or chemical toxicity, comprising administering a therapeutically effective amount of a PAR-1 inhibitor.

The present invention also provides methods for reducing fibroproliferative tissue effects in a patient that will be exposed, is concurrently exposed, or was exposed to radiation and/or chemical toxicity, comprising administering a therapeutically effective amount of a PAR-1 inhibitor.

In one embodiment of any of the methods detailed above, the PAR-1 inhibitor is administered in an amount sufficient to maintain the patient's plasma level of the PAR-1 inhibitor at or above 1 μM for 24 hrs.

The present invention also provides methods useful for reducing lethality or other adverse pathophysiological effects in a patient after non-therapeutic radiation and/or chemical exposure comprising administering a therapeutically effective amount of a protease activated receptor-1 (PAR-1) inhibitor.

### DETAILED DESCRIPTION OF THE DRAWINGS

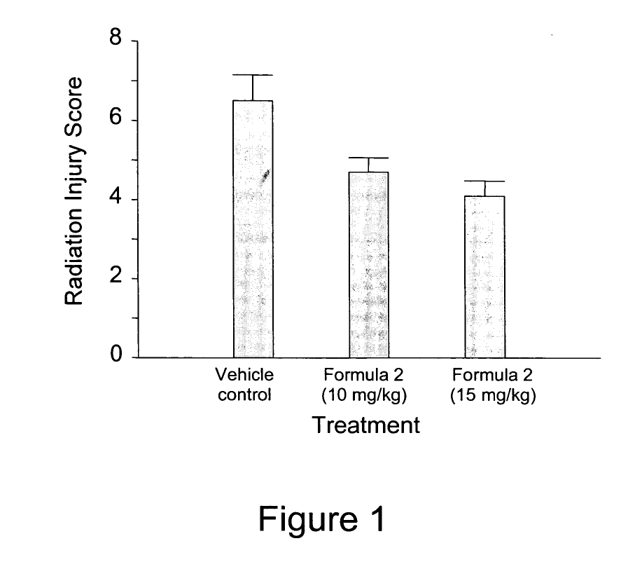

FIG. 1 illustrates the radiation injury score in irradiated rats administered one of three different treatments: (i) vehicle control; (ii) 10 mg/kg/day of Formula 2; or (iii) 15 mg/kg/day of Formula 2.

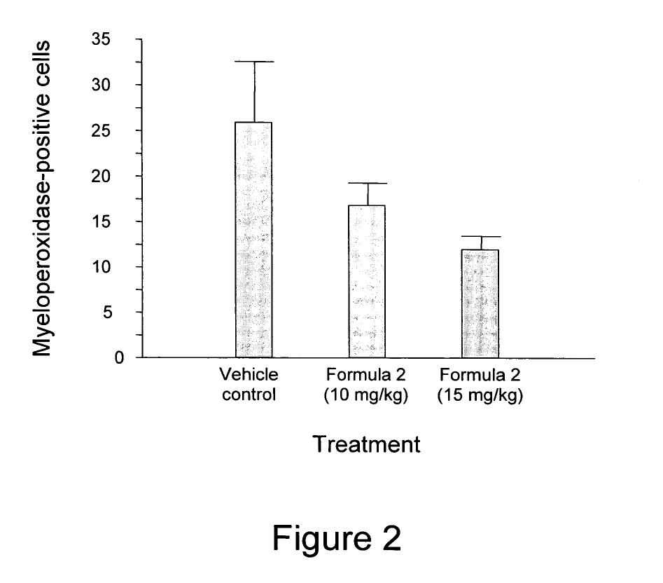

FIG. 2 illustrates neutrophil infiltration (as assayed by myeloperoxidase-positive cells) in irradiated rats administered one of three different treatments: (i) vehicle control; (ii) 10 mg/kg/day of Formula 2; or (iii) 15 mg/kg/day of Formula 2.

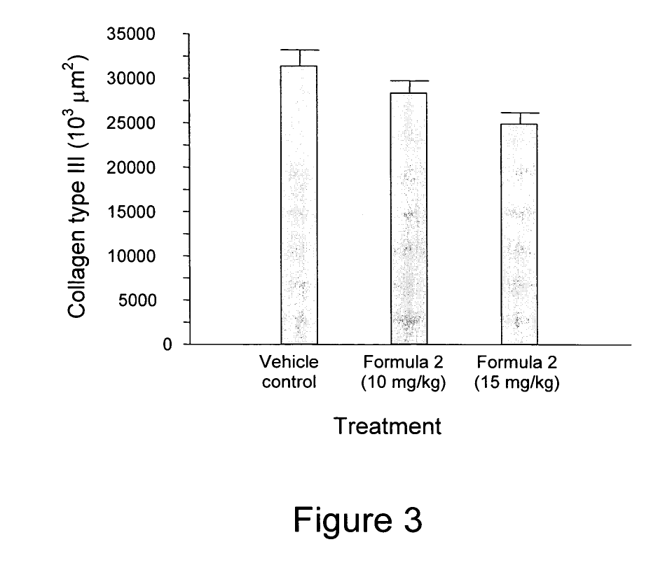

FIG. 3 illustrates collagen type III deposition in irradiated rats administered one of three different treatments: (i) vehicle control; (ii) 10 mg/kg/day of Formula 2; or (iii) 15 mg/kg/day of Formula 2.

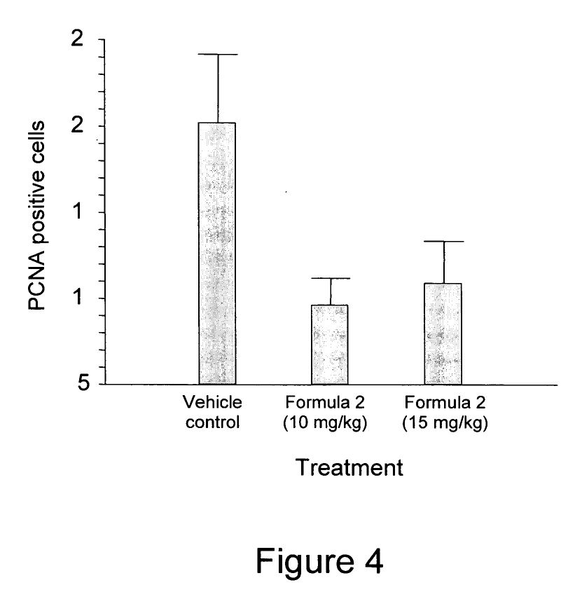

FIG. 4 illustrates smooth muscle cell proliferation (as assayed by proliferation cell nuclear antigen (PCNA) positive cells) in irradiated rats administered one of three different treatments: (i) vehicle control; (ii) 10 mg/kg/day of Formula 2; or (iii) 15 mg/kg/day of Formula 2.

### DETAILED DESCRIPTION OF THE INVENTION

As used herein, the following terms shall have the definitions set forth below.

As used herein, the phrases “radiation toxicity” or “radiation-induced toxicity” refer to radiation-induced injury to a cell or tissue arising from exposure to radiation. Phenotypically, radiation-induced injury includes one or more of the following: structural radiation injury to a cell or tissue, increased neutrophil infiltration, increased collagen type III deposition, and increased smooth muscle cell proliferation relative to that seen in a cell or tissue not exposed to radiation.

As used herein, the phrase “chemical-induced toxicity” refers to chemical-induced injury to a cell or tissue arising from exposure to a chemical. Phenotypically, chemical-induced injury includes one or more of the following: structural chemical injury to a cell or tissue, inflammation, fibroproliferative tissue effects, adverse tissue remodeling, (e.g., increased neutrophil infiltration), relative to that seen in a cell or tissue not exposed to a chemical.

As used herein, the phrase “protease activated receptor-1 inhibitor” also referred to herein as “PAR-1,” means an agent that inhibits signaling from protease activated receptor-1. An exemplary assay for identifying PAR-1 inhibitors (filtration binding assay) is described in Ahn et al., Mol Pharnnacol, 51:350-356 (1997). Briefly, human platelet membranes (40 micrograms/0.2 mL reaction mixture) were incubated with 10 nM [3H]haTRAP and various concentrations of test compound at room temperature for 1 hour. Bound and free radioactivity were separated by rapid vacuum-assisted filtration and bound radioactivity was quantified by liquid scintillation counting. Curve fitting was performed and the concentration of test compound to displace 50% of specific binding was determined.

As used herein, the phrase “therapeutically effective amount” with respect to a PAR-1 inhibitor used to treat and/or prevent radiation-induced toxicity means an amount which provides a therapeutic benefit to reduce radiation-induced toxicity by 15% or more as measured by Radiation Injury Score. Similarly, the phrase “therapeutically effective amount” with respect to a PAR-1 inhibitor used to treat and/or prevent chemical-induced toxicity means an amount which provides a therapeutic benefit to reduce chemical-induced toxicity by 15% as measured by structural damage to a cell or tissue (e.g., presence, size, or duration of structural damage) or neutrophil infiltration.

As used herein the phrase “pharmaceutically acceptable salt” refers to a non-toxic salt prepared from a pharmaceutically acceptable acid or base (including inorganic acids or bases, or organic acids or bases). Examples of such inorganic acids are hydrochloric, hydrobromic, hydroiodic, sulfuric, and phosphoric. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. Examples of such inorganic bases include metallic salts made from aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylgulcaine), lysine, and procaine.

As used herein the phrase “radiation-response modifier” refers to a compound that improves the radiation response and survival in patients (i.e., reduces the effects of radiation exposure).

Preferably, assuming a patient having a bodyweight of 70 kg, an exemplary dosing regimen for a PAR-1 inhibitor is QD: up to 4000 mg. For example, a preferred dosing regimen for a PAR-1 inhibitor (e.g., Formula 2) is as follows, QD: 900 mg to 4000 mg, more preferably 2400 mg; BID: 284 mg to 392 mg, more preferably 338 mg; or TID: 224 mg to 352 mg, more preferably 288 mg. Preferably, the dosing regimen maintains the patient's plasma level of PAR-1 inhibitor at or above 1 μM for 24 hrs.

The dosing regimen for a PAR-1 inhibitor may be administered by various routes including but not limited to, oral (p.o.), intraperitoneal (i.p.), intravascular (i.v.), subcutaneous (s.c.), or intrathecal (i.t.) routes of administration.

The amount and frequency of administration of the compounds of the invention and/or the pharmaceutically acceptable salts thereof will be regulated according to the judgment of the attending clinician considering such factors as age, condition and size of the patient as well as severity of the symptoms being treated.

In another embodiment, the PAR-1 inhibitor is selected from the group consisting of Formula 1, Formula 2, Formula 3, Formula 4, Formula 5, BMS-200261 (Bernatowicz et al., 39(25):4879-4887 (1996)), RWJ-56110 (Maryanoff et al., Curr Med Chem Cardiovasc Hematol Agents 1(1): 13-36 (2003)), and RWJ-58259 (Maryanoff et al., Curr Med Chem Cardiovasc Hematol Agents, 1(1):13-36 (2003)), a blocking antibody to PAR-1 (Kahn et al., Clin Invest, 103(6):879-887 (1999)), a pepducin to PAR-1 (Covic et al., Proc Natl Acad Sci USA 99(2):643-648 (2002), an antisense oligonucleotide to PAR-1, a small interfering RNA or a short hairpin RNA to the mRNA encoding PAR-1, or a pharmaceutically acceptable salt thereof, or a combination of two or more of the above.

Mucositis is a process that progresses in five phases as detailed below. Phase 1, “the initial phase,” includes: DNA strand breaks, and reactive oxygen species generation. Phase 2, “the primary damage response phase” includes: activation of NF-kB and p53 pathway; NF-κB up-regulation of genes that may exert an effect on mucosal toxicity, including apoptosis-regulating genes of the BCL2 family; up-regulation of c-Jun and c-Jun amino-terminal kinase, which in turn up-regulates NRF2; and production of proinframmatory cytokines, TNF-alpha, IL- ibeta, IL-6, the presence of which may cause damage to epithelium via reduced oxygenation and basal cell death, endothelium, and connective tissue; radiation and some cytotoxic agents also cause apoptosis via hydrolyzation of sphingomyelin (a cell-membrane lipid), a process that increases ceramide levels and results in cell apoptosis; fibroblasts in the submucosa may be damaged by radiation or chemotherapy, either directly or via stimulation of metalloproteinases. Phase 3, “the signal amplification phase,” includes: a range of proteins that accumulate and target the submucosa, causing tissue damage and initiating a positive feedback loop, amplifying the primary damage caused by the radiation or chemotherapy. For example, a pathway that results in cell death is activated by TNF-α, which in turn activates NFκB and initiates mitogen-activated protein kinase (MAPK) signaling, in turn activating JNK (a member of the MAP kinase family), in turn regulating the activity of AP1. Cell death caused by this pathway occurs in the submucosa as well as the epithelium. TNF-α and IL-1β both induce matrix metalloproteinase activation. Phase 4, “the ulcerative phase,” may include: functional trauma caused lesions (e.g., with respect to oral mucositis, the lesions appear in the mouth); excessive bacterial colonization of lesions, (e.g., with respect to oral mucositis, the bacterial colonization of lesions may be exacerbated by reduced salivary levels and poor oral hygiene as often happens in neutropenic patients); endotoxin released from gram-negative organisms and cell wall products from gram-positive bacteria may then interact with tissue macrophages to trigger release of further IL-1 and TNF-α, exacerbating mucosal damage. Secondary infections that result include fungal infections, viral infections and bacterial infections. Phase 5, “the healing phase,” includes: cell proliferation and differentiation returns to normal; bone marrow recovery results in increased numbers of white cells and control of local infection.

An exemplary assay for the treatment of oral mucositis may be performed as described in the phase 3 clinical trial of Kepivance™ (palifermin) (see, Spielberger, N Engl J Med, 351(25):2590-2598 (2004)).

Encompassed within the scope of the present invention are methods for treating, ameliorating, and/or preventing mucositis (e.g., oral mucositis) caused by radiation- and/or chemical-induced toxicity in non-malignant tissue in a patient comprising administering a therapeutically effective amount of a protease activated receptor-1 (PAR-1) inhibitor. For example, wherein the PAR-1 inhibitor is:

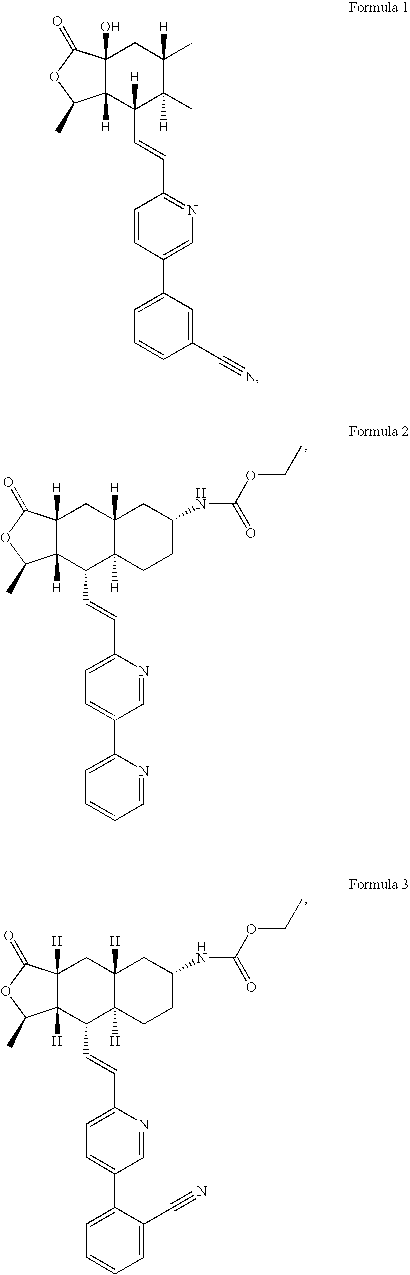

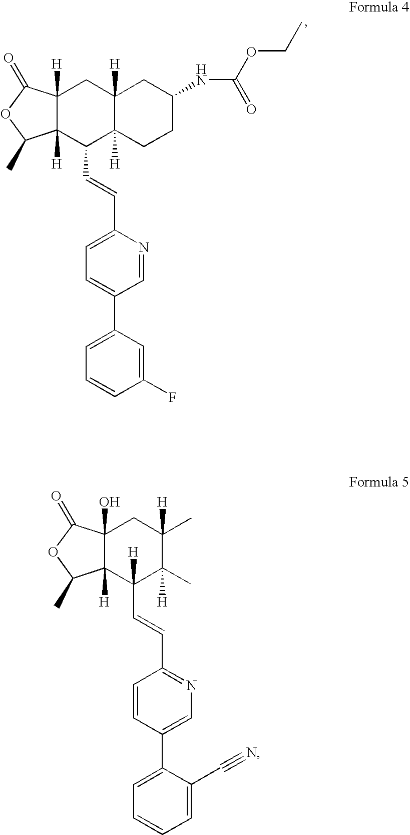

BMS-200261, RWJ-56110, RWJ-58259, a blocking antibody to PAR-1, a pepducin to PAR-1, an antisense oligonucleotide to PAR-1, a small interfering RNA or a short hairpin RNA to the mRNA encoding PAR-1, or a pharmaceutically acceptable salt thereof, or a combination of two or more of the above.

Also encompassed within the scope of the present invention are methods for treating, ameliorating, and/or preventing mucositis (e.g., oral mucositis) caused by radiation- and/or chemical-induced toxicity in non-malignant tissue in a patient comprising administering one or more PAR-1 inhibitors in combination with KepivanceTM (palifermin) (Spielberger, N Engl JMed, 351(25):2590-2598 (2004)), amifostin (Dunst et al., Strahlenther Onkol, 176(9):416-421 (2000)), L-glutamine (Blijlevens et al., Support Care Cancer, 13(10):790-796 (2005); and Aquino et al., Bone Marrow Transplant, 36(7):611-616 (2005)), teduglutide (Booth et al., Cell Prolif, 37(6):385-400 (2004)), sucralfate mouth rinses (Makkonen et al., Int J Radiat Oncol Biol Phys, 30(1):177-182 (1994)), iseganan (Cole and Waring, Am J Respir Med, 1(4):249-259 (2002)), lactoferrin (Van't Land et al., Dig Dis Sci, 49(3):425-433 (2004)), mesna (Ypsilantis et al., J Surg Res, 121(1):84-91 (2004)), trefoil factor (Beck et al., Gastroenterology, 126(3):796-808 (2004); and Xian et al., Am J Physiol, 277(4 Pt 1):G785-975 (1999)), or a combination of two or more of the above.

In addition, encompassed within the scope of the present invention are methods for treating, ameliorating, and/or preventing mucositis (e.g., oral mucositis) caused by radiation- and/or chemical-induced toxicity in non-malignant tissue in a patient comprising administering one or more PAR-1 inhibitors in combination with another radiation-response modifier.

### EXPERIMENTS

Intestinal fibrosis

A particularly relevant animal model for radiation toxicity is described in Wang et al., J Thromb Haemost, 2(11):2027-2035 (2004)). In brief, a “scrotal hernia” containing a 4 cm loop of distal ileum is surgically created in male Sprague-Dawley rats. After a 3 week recovery period, the scrotal hernia is irradiated locally without exposing the rest of the animal to ionizing radiation.

Intestinal fibrosis—Experiment 1

Scrotal hernias were created in rats which subsequently received one of three different treatments subcutaneously: (i) vehicle control (i.e., 0.4% methyl cellulose); (ii) 10 mg/kg/day of Formula 2; and (iii) 15 mg/kg/day of Formula 2. The treatments were administered for 24 days total, starting the day before irradiation (i.e., from Day-1 to Day 23). Beginning on Day 1, the scrotal hernia of each animal was irradiated locally by exposure to 5 Gy for 9 days. After an additional 2 week observation period following treatment, the rats were euthanized and assessed for radiation toxicity using these endpoints: structural radiation injury, immunohistochemistry (e.g., neutrophil infiltration, collagen type III deposition, smooth muscle cell proliferation, extracellular matrix-associated TGF-β immunoreactivity, collagen type I deposition, macrophages (ED-2)), and morphomety.

Structural Radiation Injury

Structural radiation injury was assessed in hematoxylin-eosin-stained sections using a radiation injury score system previously described (see, Langberg et al., Acta Oncol, 31(7):781-787 (1992); and Hauer-Jensen et al., Acta Radiol Oncol, 22(4):299-303 (1983)). In brief, seven parameters of radiation injury (mucosal ulcerations, epithelial atypia, thickening of subserosa, vascular sclerosis, intestinal wall fibrosis, ileitis cystica profunda, and lymph congestion) were graded (0-3) according to severity. The sum of the scores for the individual alterations constitutes the Radiation Injury Score. All specimens were evaluated by two separate researchers and non-concordant scores were resolved by consensus.

As illustrated in FIG. 1, structural radiation injury was examined in irradiated rats that were administered one of three different treatments: (i) vehicle control; (ii) 10 mg/kg/day of Formula 2; or (iii) 15 mg/kg/day of Formula 2. In short, structural radiation injury as measured by Radiation Injury Score was less in animals treated with Formula 2 as compared to animals treated with vehicle (p=0.003 per the Jonckheere-Terpstra test).

Immunohistochemistry

Quantitative immunohistochemistry was used to determine: (i) neutrophil infiltration by mycloperoxidase stalninig; (ii) intestinal smooth muscle cell proliferation using proliferation cell nuclear antigen (PCNA) labeling index; (iii) collagen deposition by staining for collagen types I and III; (iv) extracellular matrix-associated transforming growth factor (TGF)-β, and (v) macrophage ED-2. Immunohistochemical staining was performed with appropriate positive and negative controls using the avidin-biotin complex (ABC) technique previously described by Wang et al., J Thromb Haemost, 2(11):2027-2035 (2004). Primary antibodies, catalog numbers, incubation times, dilutions, and companies were: polyclonal antimyeloperoxidase antibody (A0398, 2 h, 1:100; Dako, Carpinteria, Calif., USA); monoclonal anti-PCNA antibody (NA03, 2 h, 1:100; Calbiochem, Cambridge, Mass., USA); polyclonal antibodies against collagen type I (1310-01, 2 h, 1:100 dilution; Southern Biotechnology Associates, Birmingham, Alab., USA); collagen type III (1330-01, 2 h, 1:100 dilution, Southern Biotechnology Associates); polyclonal rabbit anti-TGF-P antibody (AB-100-NA, 2 h, 1:300 dilution; R&D, Minneapolis, Minn., USA); and ED-2 (MCA342, 2 h, 1:100 dilution, Serotec, Rahway, N.C., USA).

Computerized image analysis was performed as previously described (see, Wang et al., Thromb Haemost, 87(1):122-128(2002); Wang et al., J Pharmacol Exp Ther, 297(1):35-42 (2001)). Neutrophil infiltration was assayed by identifying myeloperoxidase-positive cells as previously described (see, Wang et al., Thromb Haemost, 87(1):122-128(2002)). Areas positive for collagen types I and III deposition were measured as previously described 'see, Raviv et al., World J Urol, 15(l1):50-55 (1997); and Wang et al., Thromb Haemost, 87(1):122-128(2002)). Extracellular matrix-associated TGF-β immunoreactivity was measured as previously described (see, Richter et al., Radiother Oncol, 39(3):243-251 (1996)).

As illustrated in FIG. 2, neutrophil infiltration (as assayed by myeloperoxidase-positive cells) was examined in irradiated rats administered one of three different treatments: (i) vehicle control; (ii) 10 mg/kg/day of Formula 2; or (iii) 15 mg/kg/day of Formula 2. In short, neutrophil infiltration was reduced in animals treated with Formula 2 as compared to animals treated with vehicle (p=0.05 per the Jonckheere-Terpstra test).

As illustrated in FIG. 3, collagen type III deposition was examined in irradiated rats administered one of three different treatments: (i) vehicle control; (ii) 10 mg/kg/day of Formula 2; or (iii) 15 mg/kg/day of Formula 2. A similar pattern was observed whereby collagen type III deposition was reduced in animals treated with Formula 2 as compared to animals treated with vehicle (p=0.005 per the Jonckheere-Terpstra test).

As illustrated in FIG. 4, smooth muscle cell proliferation (as assayed by proliferation cell nuclear antigen (PCNA) positive cells) was examined in irradiated rats administered one of three different treatments: (i) vehicle control; (ii) 10 mg/kg/day of Formula 2; or (iii) 15 mg/kg/day of Formula 2. In short, smooth muscle cell proliferation was reduced in animals treated with Formula 2 as compared to animals treated with vehicle (p=0.04 per the Jonckheere-Terpstra test).

Likewise, quantitative immunohistochemical analyses revealed a trend toward increased levels of collagen type I deposition, extracellular matrix-associated TGF-P, and macrophages (i.e., ED-2) in vehicle-treated animals as compared to animals treated with Formula 2 (p=0.4, 0.1, 0.4, respectively, per the Jonckheere-Terpstra test).

Morphometry

The thickness of the intestinal wall proper (submucosa, muscularis externa, and subserosa, but excluding the mucosa) was measured with an eyepiece linear microruler. Five measurements, 500 μm apart, were obtained, averaged for each specimen, and used as a single value for statistical calculations. Notably, there was a trend toward intestinal wall thickening in vehicle-treated animals as compared to animals treated with Formula 2 (p=0.2 per the Jonckheere-Terpstra test).

The surface area of the intestinal mucosa was measured in vertical sections using a projection/cycloid method previously described (see Baddeley et al., J Microsc, 142(Pt 3):259-276 (1986); and Langberg et al., Acta Oncol, 35(1):81-87 (1996)). This technique does not require. assumptions about the shape or orientation distribution of the specimens and thus circumvents problems associated with other similar procedures for surface area measurement. Similarly, there was a trend toward serosal thickening in vehicle-treated animals as compared to animals treated with Formula 2 (p=0.3 per the Jonckheere-Terpstra test).

Statistical Methods

Differences in endpoints as a function of drug treatment (PAR-1 inhibitor vs. vehicle) were assessed using fixed-factor analysis of variance and post hoc comparisons with Newman-Keul's test (NCSS2000 for Windows 95, NCSS, Kaysville, Utah, USA). Univariate comparisons were performed with the Mann-Whitney U-test using StatXact 5 (Cytel Software, Cambridge, Mass., USA), a software package for exact non-parametric inference.

Intestinal Fibrosis—Experiment 2

Scrotal hernias are created in rats which subsequently receive one of three different treatments: (i) vehicle control; (ii) 10 mg/kg/day of Formula 2; and (iii) 15 mg/kg/day of Formula 2. The treatments start the day before irradiation (i.e., Day-1) and are administered subcutaneously for 24 days followed by administration in the chow for the next 168 days (i.e., s.c. administration from Day-1 to Day 23, followed by p.o. administration from Day 24 to Day 191). On Day 1, the scrotal hernia of each animal is irradiated locally by exposure to 5 Gy for 9 days. After an additional 2 week observation neriod following treatment, the rats are euthanized and assessed for radiation toxicity using endpoints such as structural radiation injury, immunohistochemistry (e.g., neutrophil infiltration, collagen type III deposition, smooth muscle cell proliferation, extracellular matrix-associated TGF-β immunoreactivity, collagen type I deposition, macrophages (ED-2)), and morphometry.

Alternatively, to compare acute to chronic radiation-induced toxicity, the animals are euthanized at 2 weeks following the last subcutaneous injection and assayed for the same endpoints.

Intestinal Fibrosis—Experiment 3

In an alternative to Experiment 2 described above, rather than administer treatments subcutaneously for the first 2 weeks, followed by administration in the chow, standard chow (for control animals) or chow with PAR-1 inhibitor Formula 2 is given solely throughout the treatment period. In addition, rather than commence administration of vehicle or PAR-1 inhibitor the day before irradiation, administration of chow containing PAR-1 inhibitor would commence 2 days before irradiation.

Additional Endpoints

Immunohistochemistry

In addition to the quantitative immunohistochemical analysis mentioned in Experiment 1, quantitative immunohistochemical analysis of TM may be performed as previously described (see, Wang et al., Am J Pathol, 160(6):2063-2072 (2002).

Similarly, qualitative immunohistochemical analysis of PAR-1 may be performed as previously described (see, Wang et al., Am J Pathol, 160(6):2063-2072 (2002).

Morphometry

In addition to the morphometric analysis mentioned in Experiment 1, morphometric analysis of radiation-induced vascular sclerosis may be performed using computer-assisted image analysis as described previously (see, Langberg et al., Acta Oncol, 35(1):81-87 (1996)). In brief, the total and luminal cross-sectional areas of submucosal vessels in the range 10-130 μm (the range of most affected by radiation) are measured (10 vessels per slide). Vessel wall ratio is calculated as the ratio between the total cross-sectional area and the vessel wall area (total cross-sectional area minus luminal cross-sectional area). The relationship between vessel wall area and total cross-sectional area is linear, and the average vessel wall ratio in each specimen is thus used as a single value for statistical purposes.

Dye Elution Method for Collagen Determination

Collagen content is determined using the dye elution method of Lopez-de Leon (Lopez-de Leon and Rojkind, J Histochem Cytochem, 33:737-747 (1985)) adapted to our model system (Langberg et al., Acta Oncol, 35:81-87 (1996)). In heterogeneous organs like intestine, the dye elution method produces more consistent data and is less influenced by changes in structures other than connective tissue compared to the more commonly used hydroxyproline assay (Hauer-Jensen et al., Acta Radiol Oncol, 25:137-142 (1986)). An additional advantage is that the method also provides direct morphologic correlates to the measured collagen content.

Fluorogenic probe reverse transcription polymerase chain reaction (RTPCR) Real-time PCR is performed according to methods detailed in Shi et al., Blood Coagul Fibrinolysis, 14:575-585 (2003)), using standard fluorogenic probe (TaqMan) technology, the ABI Prism 7000 Sequence Detection System, TaqMan Universal PCR MasterMix, and appropriate Assays-on-Demand Gene Expression kits for from PE Applied Biosystems (Foster City, Calif.). Relative quantitation of mRNA species will be performed using the comparative threshold cycle (CT) method (see, e.g., PE Applied Biosystems. Relative quantitation of gene expression (see, e.g., Norwalk, Conn.: Perkin-Elmer Corp., 2001). Fluroogenic probe (LCM) RT-PCR to detect mRNA of PAR-1 is performed as previously described (see, Wang et al., Am J Pathol, 160(6):2063-2072 (2002). Likewise, fluroogenic probe (LCM) RT-PCR to detect mRNA of TGF-β1, procollagen types I and III, and other relevant transcripts may be performed in a similar manner.

In-Situ Hybridization

In situ hybridization of PAR-1 may be performed as previously described (see, Wang et al., Am J Pathol, 160(6):2063-2072 (2002).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Various publications are cited herein, the disclosures of which are incorporated by reference in their entireties.

What is claimed:

1. A method for treating and/or preventing radiation- and/or chemical-induced toxicity in non-malignant tissue in a patient comprising administering a therapeutically effective amount of a protease activated receptor-1 (PAR-1) inhibitor.

2. The method of claim 1, wherein the PAR-1 inhibitor is: 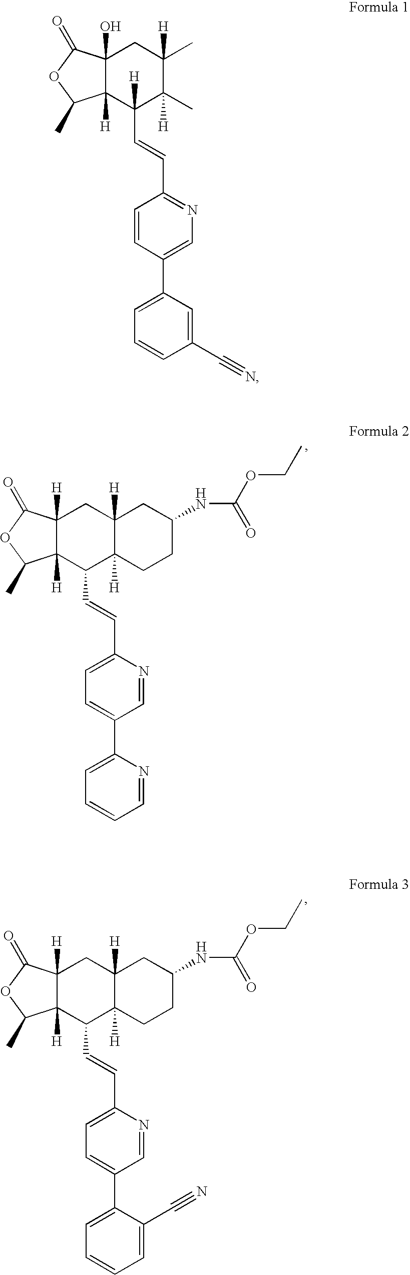 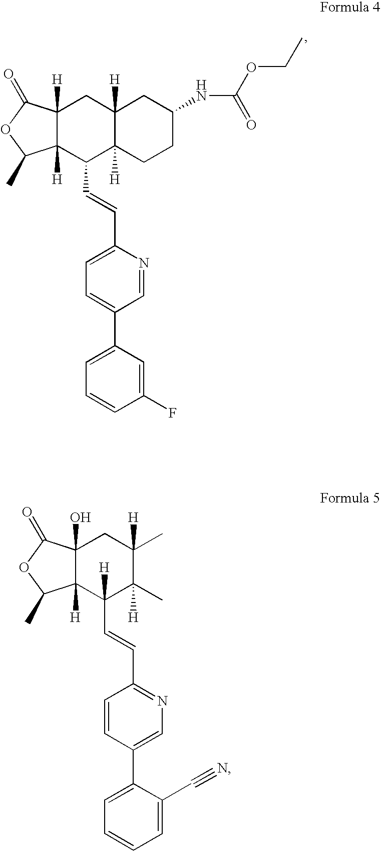
BMS-200261, RWJ-561 10, RWJ-58259, a blocking antibody to PAR-1, a pepducin to PAR-1, an antisense oligonucleotide to PAR-1, a small interfering RNA or a short hairpin RNA to the mRNA encoding PAR-1, or a pharmaceutically acceptable salt thereof, or a combination of two or more of the above.

3. The method of claim 1, wherein the PAR-1 inhibitor is: 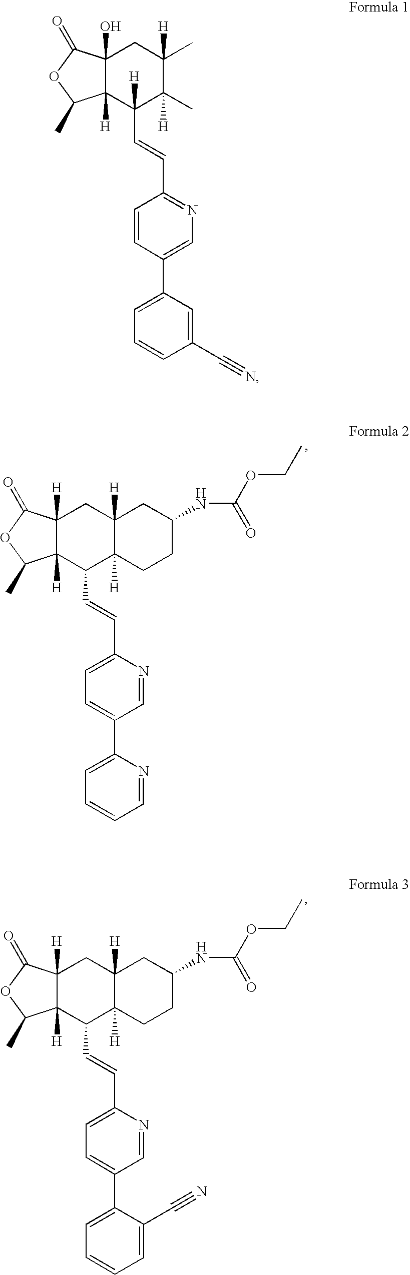 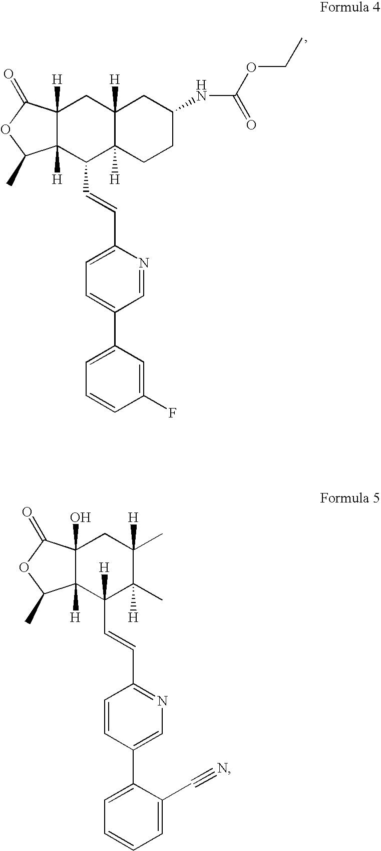
or a pharmaceutically acceptable salt thereof, or a combination of two or more of the above.

4. The method of claim 1, wherein the PAR-1 inhibitor is Formula 2, or a pharmaceutically acceptable salt thereof.

5. The method of claim 1, wherein the radiation- and/or chemical-induced toxicity is one or more of intestinal fibrosis, pneumonitis, and mucositis.

6. The method of claim 1, wherein the radiation- and/or chemical-induced toxicity is intestinal fibrosis.

7. The method of claim 1, wherein the radiation- and/or chemical-induced toxicity is oral mucositis.

8. The method of claim 1, wherein the radiation- and/or chemical-induced toxicity is intestinal mucositis, intestinal fibrosis, intestinal radiation syndrome, or pathophysiological manifestations of intestinal radiation exposure.

9. The method of claim 1, wherein the PAR-1 inhibitor is administered in combination with Kepivance™ (palifermin), L-glutamine, teduglutide, sucralfate mouth rinses, iseganan, lactoferrin, mesna, trefoil factor, or a combination of two or more of the above.

10. The method of claim 1, wherein the PAR-1 inhibitor is administered in combination with another radiation-response modifier.

11. A method for reducing structural radiation injury in a patient that will be exposed, is concurrently exposed, or was exposed to radiation and/or chemical toxicity, comprising administering a therapeutically effective amount of a PAR-1 inhibitor.

12. A method for reducing inflammation in a patient that will be exposed, is concurrently exposed, or was exposed to radiation and/or chemical toxicity, comprising administering a therapeutically effective amount of a PAR-1 inhibitor.

13. A method for reducing adverse tissue remodeling in a patient that will be exposed, is concurrently exposed, or was exposed to radiation and/or chemical toxicity, comprising administering a therapeutically effective amount of a PAR-1 inhibitor.

14. A method for reducing fibroproliferative tissue effects in a patient that will be exposed, is concurrently exposed, or was exposed to radiation and/or chemical toxicity, comprising administering a therapeutically effective amount of a PAR-1 inhibitor.

15. The method of any one of claims 1, 11, 12, 13, or 14, wherein the PAR-1 inhibitor is administered in an amount sufficient to maintain the patient's plasma level of the PAR-1 inhibitor at or above 1 μM for 24 hrs.

16. A method for reducing lethality or other adverse pathophysiological effects in a patient after non-therapeutic radiation and/or chemical exposure comprising administering a therapeutically effective amount of a protease activated receptor-1 (PAR-1) inhibitor.
